SEQUENCE LISTING (37 sequences)

SEQ ID NO: 1 - DNA, 21 bp, Artificial Sequence
  Synthetic sgRNA
gcctacccccctgcgcgccga

SEQ ID NO: 2 - DNA, 21 bp, Artificial Sequence
  Synthetic sgRNA
gttcgcgtagacaggcgcttc

SEQ ID NO: 3 - DNA, 20 bp, Artificial Sequence
  Synthetic U6 promoter forward primer
gagggcctatttcccatgat

SEQ ID NO: 4 - DNA, 20 bp, Artificial Sequence
  Synthetic ADRA2B
gctgtggtcattggcgtttt

SEQ ID NO: 5 - DNA, 20 bp, Artificial Sequence
  Synthetic ADRA2B
gcggaagtcctggttgaaga

SEQ ID NO: 6 - DNA, 20 bp, Artificial Sequence
  Synthetic ANXA2
aatcctgtgcaagctcagct

SEQ ID NO: 7 - DNA, 20 bp, Artificial Sequence
  Synthetic ANXA2
tgctgcggttggtcaaaatg

SEQ ID NO: 8 - DNA, 20 bp, Artificial Sequence
  Synthetic CCNA1
ggtcccgatgcttgtcagat

SEQ ID NO: 9 - DNA, 20 bp, Artificial Sequence
  Synthetic CCNA1
ctttccagctggagggaagg

SEQ ID NO: 10 - DNA, 20 bp, Artificial Sequence
  Synthetic CDK2
cattcctcttcccctcatca

SEQ ID NO: 11 - DNA, 20 bp, Artificial Sequence
  Synthetic CDK2
cagggactccaaaagctctg

SEQ ID NO: 12 - DNA, 20 bp, Artificial Sequence
  Synthetic CDK4
agctcccgaagttcttctgc

SEQ ID NO: 13 - DNA, 20 bp, Artificial Sequence
  Synthetic CDK4
catctcgaggccagtcatcc

SEQ ID NO: 14 - DNA, 20 bp, Artificial Sequence
  Synthetic EIF1
tgtccaagggatcgctgatg

SEQ ID NO: 15 - DNA, 20 bp, Artificial Sequence
  Synthetic EIF1
cagctgatcgtccttagcca

SEQ ID NO: 16 - DNA, 20 bp, Artificial Sequence
  Synthetic ELM02
tttgccctccaaacccaact

SEQ ID NO: 17 - DNA, 20 bp, Artificial Sequence
  Synthetic ELM02
caaggatctcgggctggatc

SEQ ID NO: 18 - DNA, 20 bp, Artificial Sequence
  Synthetic ER01La
tgaagaggccgtgtcctttc

SEQ ID NO: 19 - DNA, 20 bp, Artificial Sequence
  Synthetic ER01La
tccactgctccaagtcgttc

SEQ ID NO: 20 - DNA, 20 bp, Artificial Sequence
  Synthetic JunD
tggaaacacccttctacggc

SEQ ID NO: 21 - DNA, 20 bp, Artificial Sequence
  Synthetic JunD
tcagcgcgtccttcttcatc

SEQ ID NO: 22 - DNA, 20 bp, Artificial Sequence
  Synthetic KIF2C
cactcgcatgtccactgtct

SEQ ID NO: 23 - DNA, 20 bp, Artificial Sequence
  Synthetic KIF2C
catctcctcgctgaccatcc

SEQ ID NO: 24 - DNA, 20 bp, Artificial Sequence
  Synthetic c-MYC
cgtcctcggattctctgctc

SEQ ID NO: 25 - DNA, 20 bp, Artificial Sequence
  Synthetic c-MYC
gcctgcctcttttccacaga

SEQ ID NO: 26 - DNA, 20 bp, Artificial Sequence
  Synthetic PEA15
ggaagacatccccagcgaaa

SEQ ID NO: 27 - DNA, 20 bp, Artificial Sequence
  Synthetic PEA15
gcacacgggttctgtagtca

SEQ ID NO: 28 - DNA, 20 bp, Artificial Sequence
  Synthetic PLCD4
ggaactctggaatgcaggct

SEQ ID NO: 29 - DNA, 20 bp, Artificial Sequence
  Synthetic PLCD4
aaagggctgatgggcttctc

SEQ ID NO: 30 - DNA, 20 bp, Artificial Sequence
  Synthetic PRDX3
gccacatgaacatcgcactc

SEQ ID NO: 31 - DNA, 20 bp, Artificial Sequence
  Synthetic PRDX3
gggagatcgttgacgctcaa

SEQ ID NO: 32 - DNA, 20 bp, Artificial Sequence
  Synthetic PTMA
cgtagacaccagctccgaaa

SEQ ID NO: 33 - DNA, 20 bp, Artificial Sequence
  Synthetic PTMA
ttcctccccaccttcttcct

SEQ ID NO: 34 - DNA, 20 bp, Artificial Sequence
  Synthetic TCF4
cgaaggaggcctcttcacag

SEQ ID NO: 35 - DNA, 20 bp, Artificial Sequence
  Synthetic TCF4
ggagctagggaaagtgctgg

SEQ ID NO: 36 - DNA, 19 bp, Artificial Sequence
  Synthetic GAPDH
gaaggtgaaggtcggagtc

SEQ ID NO: 37 - DNA, 20 bp, Artificial Sequence
  Synthetic GAPDH
gaagatggtgatgggatttc

(19) United States
(12) Patent Application Publication
(10) Pub. No.: US 2022/0002816 A1
(43) Pub. Date: Jan. 6, 2022

(54) IDENTIFICATION OF JUND TARGET GENES FOR INHIBITION OF PROSTATE CANCER CELL GROWTH

(71) Applicant: Center for Cancer Research & Therapeutic Development, Clark Atlanta University, Atlanta, GA (US)

(72) Inventors: Bethtrice THOMPSON-ELLIOTT, Kennesaw, GA (US); Shafiq Ahmed KHAN, Mableton, GA (US); Ana Cecilia MILLENA, Austell, GA (US)

(21) Appl. No.: 17/293,003

(22) PCT Filed: Oct. 31, 2019
(86) PCT No.: PCT/US2019/059216

§ 371 Date: May 11, 2021

Related U.S. Application Data
(60) Provisional application No. 62/760,378, filed on Nov. 13, 2018.

(51) Int. Cl. C12Q 1/6886; C12N 15/113; A61P 35/00; A61K 31/551

(52) U.S. Cl. CPC C12Q 1/6886; C12N 15/113; C12Q 2600/158; A61K 31/551; C12N 2310/14; A61P 35/00

(57) ABSTRACT

This disclosure generally relates to the development of therapeutic and chemo-preventative strategies to prostate cancer initiation and carcinogenesis. Specifically, the disclosure relates to the role the JunD transcription factor plays in the deregulation of cell proliferation. The disclosure relates to a novel method of identifying JunD target genes and inhibiting the expression and/or function of the JunD target genes to interfere with the regulation of cancer cell proliferation and early stages of carcinogenesis.

### STATEMENT REGARDING FEDERALLY SPONSORED RESEARCH OR DEVELOPMENT

Development of the inventions described herein was at least partially funded with government support under grant numbers 5G12MD007590 and 2P20MD002285 awarded by the National Institutes of Health. The U.S. government has certain rights in the invention.

### FIELD OF THE INVENTION

The present disclosure generally relates to the development of therapeutic and chemo-preventative strategies to prostate cancer initiation and carcinogenesis. Specifically, the disclosure relates to the role the JunD transcription factor plays in the deregulation of cell proliferation. The disclosure relates to a novel method of identifying JunD target genes and inhibiting the expression and/or function of the JunD target genes to interfere with the regulation of cancer cell proliferation and early stages of carcinogenesis.

### BACKGROUND

Prostate cancer is the most prevalent malignancy in men worldwide and remains a frequent cause of cancer-related deaths in men. As a result of increasing age linking the likelihood of developing prostate cancer and the likelihood of developing advanced metastatic prostate cancer in men of African descent, improvements in early detection and advances in prostate cancer therapeutics are increasing [2,4-8]. Despite recent breakthroughs in identifying specific prostate cancer genes, the molecular mechanisms involved in the initiation and progression of prostate cancer are not yet clearly understood.

The development of prostate cancer occurs with uncontrolled cell proliferation which leads to development of benign low-grade prostatic intraepithelial neoplasia (LGPIN), followed by high-grade prostatic intraepithelial neoplasia (HGPIN), a precursor for invasive carcinoma, which leads to the development of highly invasive intraductal carcinoma [14, 15]. The initiation of carcinogenesis in the prostate is primarily dependent on deregulation of genes that control cell proliferation and as a result causes either a loss of inhibitory controls of cell cycle progression or an upregulation of factors which stimulate cell proliferation [16-18].

Transcription factors (TFs) have been implicated as important drivers of prostate cancer, primarily due to their overexpression in cell lines and/or patients' prostate cancer tissue samples. Examples include c-MYC, ETS, GATA2, and E2F3 [9, 11-12, 19-24]. Members of the activating protein-1 (AP-1) transcription factor family are often implicated as oncogenic cancer drivers [20, 25-29]. The AP-1 transcription factor is composed of dimer combinations primarily formed between Jun (JunB, c-Jun, and JunD) and Fos (FosB, c-Fos, Fra1, and Fra2) protein family members [29-30]. Jun proteins form homo (Jun-Jun) or heterodimers (Jun-Fos), while Fos proteins can only form heterodimers with Jun proteins that can bind to the TPA-response element (TRE) or cyclic AMP-responsive elements (CRE) in the promoter regions of target genes [20, 29-30]. AP-1 activity is modulated through its dimer composition which leads to differential transcriptional and biological functions [20]. AP-1 regulates cellular proliferation, survival, apoptosis, inflammation, differentiation, locomotion, and plays a central role in oncogenesis [20, 28-29]. The AP-1 transcription factors and their upstream kinases have been implicated in prostate cancer initiation and progression [31-33]. For example, c-Jun or c-Fos overexpression increases cell proliferation and invasiveness of prostate cancer cell lines [34]. Furthermore, high levels of these proteins are associated with prostate cancer disease recurrence [33]. Previous studies also indicate that JunD along with Fra1 and Fra2 are essential in prostate cancer proliferation and confers protection against radiation-induced cell death [35]. JunD has also been shown to be required for cell proliferation of prostate cancer cells, while c-Jun and JunB had no effect on cell proliferation [29].

c-MYC, an oncogenic TF, is involved in regulating several biological activities including cell proliferation, apoptosis, and also carcinogenesis [36-40]. c-MYC protein has been found to be overexpressed in several cancers including prostate cancer, but in normal (non-transformed) cells, c-MYC expression levels are low and its function is tightly regulated by developmental or mitogenic signals [11, 36-37, 40-42]. c-MYC also regulates the cell cycle and cell metabolisms. Its levels accumulate as the initial response gene and are maintained at high levels throughout the cell cycle in the presence of growth factors [19, 43]. In the presence of mutations, c-MYC levels become out of control thereby leading to tumorigenesis [19, 40]. In-depth analyses of c-MYC normal function as well as its overexpression leading to carcinogenesis have been reported [29], but its regulation is not well understood. In the absence of JunD protein in prostate cancer cells, cell proliferation is inhibited along with a significant decrease in proteins involved in cell cycle regulation including c-MYC [29]. Furthermore, overexpression of JunD significantly increases cell proliferation and colony formation in prostate cancer cells [29].

There remains a need to understand the role of JunD (as a part of AP-1 TF) in the regulation of the expression of genes, and in particular to determine if cell cycle can be inhibited through a decrease in JunD protein levels, thereby decreasing the progression of prostate cancer.

### SUMMARY

JunD, a member of the AP-1 family, is essential for cell proliferation in prostate cancer cells. The present disclosure identifies the essential role JunD knock-down plays in distinct gene and protein expression patterns. Provided herein are methods for inhibiting prostate cancer cell growth comprising identifying JunD target genes, decreasing the expression of said target genes, and/or inhibiting the function of the JunD target genes to interfere with the regulation of cancer cell proliferation and early stages of carcinogenesis.

Overexpression of JunD induces the expression of the JunD target genes including c-MYC. JunD is a key regulator of cell cycle progression. Therefore, inhibiting its target genes described herein provides an effective approach to block prostate cancer carcinogenesis.

In one aspect, the method identifies the products of JunD target genes which target MYC as the key downstream regulator. In another aspect, the method utilizes the cell cycle control/regulation pathway to induce decreases in gene expression in PRDX3, PEA15, KIF2C and CDK2 following JunD knockdown.

Additional advantages will be set forth, in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. Like numbers represent the same elements throughout the figures. The drawing figures are not necessarily to scale and certain features may be shown exaggerated in scale or in a somewhat generalized or schematic form in the interest of clarity and conciseness. For more complete understanding of the features and advantages of the present invention, reference is now made to the detailed description of the invention along with the accompanying figures.

FIG. 1A is a graph depicting the generation of PC3 cells JunD knock-out (KO) cells by CRISPR/Cas9 genome editing and confirming JunD KO in PC3 cells by Western Blot analysis (inset).

FIG. 1B is a graph showing the results of cell proliferation assays performed to measure cell growth rate and growth curve (1-8 days) of PC3-JunD depleted cells (JunD sg1/2-1) and PC3 wt (control cells).

FIG. 1C is a illustration of cell size determination by Image J and visualization by staining cells with Dapi (nuclei) and Phalloidin (actin filaments).

FIG. 2 is a schematic drawing depicting microarray and proteomic analyses of PC3 JunD knockdown siControl lysates, wherein differentially expressed molecules were selected based on fold change in their expression; p<0.05.

FIG. 3A is a Venn diagram depiction of the at least 2 fold comparison in PC3 knockdown compared with siControl cells.

FIG. 3B depicts a heatmap of differentially expressed genes (F.C=1.1, p<0.01).

FIG. 3C illustrates an RNA microarray analysis showing the top 10 down regulated genes in PC3-JunD deficient cells and the role of the genes in cell cycle regulation.

FIG. 3D is a graph depicting Ingenuity Pathway Analysis (IPA) of cell cycle control/regulation pathway for members exhibiting gene expression down-regulation after JunD knock-down in PC3 cells.

FIG. 3E is a graph depicting IPA upstream pathway analysis of mass spectrometry proteomic data of MYC as an upstream regulator of JunD targets.

FIG. 3F depicts IPA upstream pathway analysis of mass spectrometry proteomic data of MYC as an upstream regulator of JunD targets.

FIG. 4A is a Venn diagram representing pair-wise comparison of microarray and proteomic analysis of molecules that were significantly down-regulated (p<0.05). The overlap represents the common molecules (115) identified by both microarray and proteomic analyses, and 76 of those genes being cell cycle-related. The top ten downregulated molecules for each category are listed below its respective group.

FIG. 4B depicts JunD targets involved in cell cycle regulation and associated with MYC pathway, confirmed by Ingenuity Pathway Analysis.

FIG. 5A is a bar graph depiction of microarray results of selected genes from JunD knockdown by qPCR analysis of PRDX3, CDK2, EIF1, KIF2C, and PEA15 gene expression after JunD knockdown in PC3 cells.

FIG. 5B is a bar graph depiction of microarray results of selected genes from JunD knockdown by qPCR analysis of PRDX3, CDK2, EIF1, KIF2C, and PEA15 gene expression after JunD knockdown in DU145 cells.

FIG. 5C is an illustration of protein levels determined by Western blot analysis.

FIG. 5D depicts a quantitative analysis of relative protein levels for PC3 and DU145 cells, wherein normalization was performed relative to the signal obtain with α-Tubulin, wherein each bar represents Mean±SEM (n=3).

FIG. 6A depicts DU145 cells overexpressing JunD (D1) and DU145 cells containing an empty vector (V6), wherein pcDNA3.1 was plated and allowed to grow for 72 hours.

FIG. 6B illustrates an increase in JunD protein levels in JunD overexpressed cells compared with control cells, wherein protein levels for c-MYC, PRDX3, KIF2C, and CDK2 were determined in total lysates by western blot analysis, wherein α-Tubulin was used as a loading control.

FIG. 6C depicts cells treated with 5 μM JQ1, a c-MYC inhibitor, and subjected to cell proliferation assays.

FIG. 6D shows an assessment of JunD target gene protein levels using western blot analysis, where α-Tubulin was used as a loading control.

FIG. 7 is an illustration of cell proliferation of PC3 and DU145 cells after transfections with control (SiControl) or PRDX3 siRNA to knock-down the expression of PRDX3, where each bar represents Mean±SEM (n=3); showing difference when compared with appropriate controls (p<0.05). Levels of PRDX3 after transfection with control siRNA and PRDX3 siRNA were determined by western blot analysis (inset).

FIG. 8A is an illustration of RNA-seq data of JunD expression between normal prostate and primary tumor samples.

FIG. 8B is a depiction of primary tumor samples with high JunD (N=71), indicated by red color and compared to the expression of JunD target genes.

FIG. 8C is a listing of percentage (%) high JunD samples with increased JunD target gene expression.

FIG. 9 is a schematic drawing indicating that JunD activates its target genes (JunD target genes), whose products target c-MYC which then leads to the activation of downstream targets (JunD/c-MYC target genes) that in turn induces cell proliferation of prostate cancer cells and carcinogenesis.

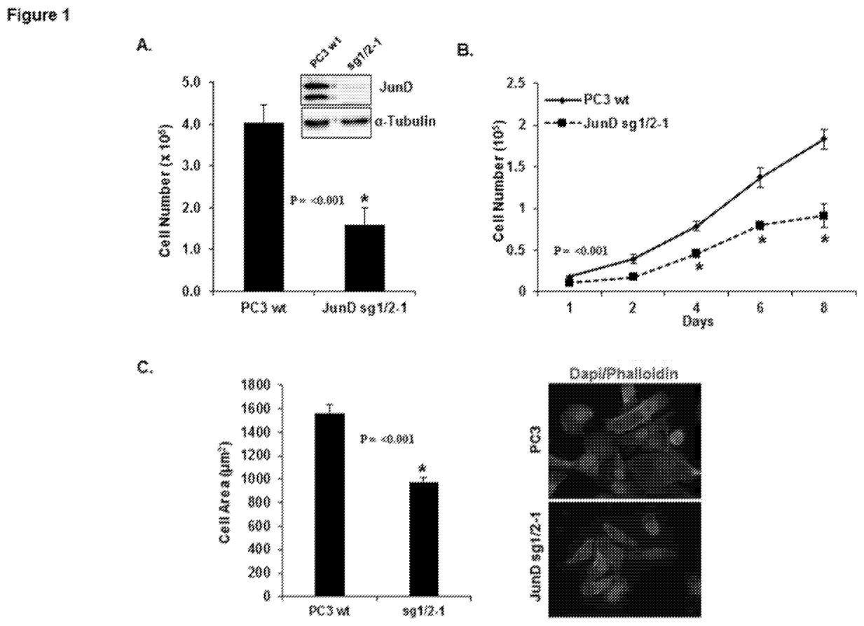

SUPPLEMENTAL FIG. 1A is an illustration validating the microarray analysis of selected genes using qPCR analysis of CCNA1, ADRA2B, PLCD4, TCF4 gene expression after JunD knockdown in PC3 cells.

SUPPLEMENTAL FIG. 1B is an illustration validating the microarray analysis of selected genes using qPCR analysis of CCNA1, ADRA2B, PLCD4, TCF4 gene expression after JunD knockdown in DU145 cells.

SUPPLEMENTAL FIG. 1C illustrates the protein levels determined by Western blot analysis.

SUPPLEMENTAL FIG. 1D is a depiction of the quantitative analysis of relative protein levels. Normalization was performed relative to the signal obtain with α-Tubulin. Each bar represents Mean±SEM (n=3). Significantly different from controls (p<0.05).

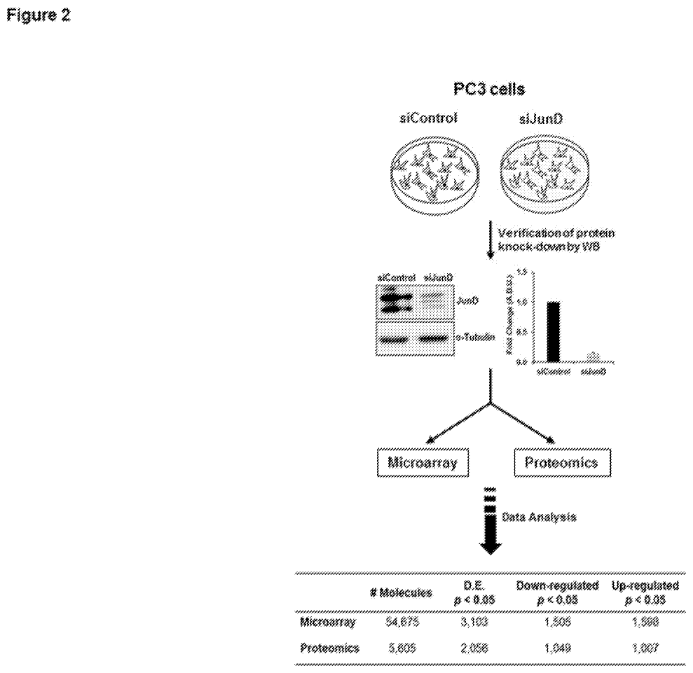

SUPPLEMENTAL FIG. 2A is an illustration validating the proteomic analysis of selected proteins using qPCR analysis of c-MYC, ANXA2, ELMO2, ERO1L-α, and PTMA gene expression after JunD knockdown in PC3 cells.

SUPPLEMENTAL FIG. 2B is an illustration validating the proteomic analysis of selected proteins using qPCR analysis of c-MYC, ANXA2, ELMO2, ERO1L-α, and PTMA gene expression after JunD knockdown in DU145 cells.

SUPPLEMENTAL FIG. 2C illustrates the protein levels determined by Western blot analysis.

SUPPLEMENTAL FIG. 2D is a depiction of the quantitative analysis of relative protein levels. Normalization was performed relative to the signal obtain with α-Tubulin. Each bar represents Mean±SEM (n=3). *Significantly different from controls (p<0.05).

### DETAILED DESCRIPTION

Although it is well established that prostate carcinogenesis results from the uncontrollable growth of cells in the prostate gland due to various factors such as inflammation, oxidative stress, DNA damage, cytokines, and certain mutations that arise that inhibit the normal gene regulation of a cell and its cell cycle, the precise molecular mechanisms involved in prostate cancer initiation and progression are not precisely known. JunD transcription factor is a key regulator of cell cycle progression. Overexpression of JunD induces the expression of the JunD target genes, including c-MYC, implicated in cell proliferation and apoptosis. Therefore, inhibiting JunD target genes described herein provides an effective approach to block prostate cancer carcinogenesis.

In one aspect, the method identifies the products of JunD target genes which target MYC as the key downstream regulator. In another aspect, the method identifies in JunD-deficient cells a decrease in protein levels in c-MYC, thereby decreasing cell proliferation in prostate cancer cells. In another aspect, the method identifies decreases in protein levels of other required proteins of cell cycle regulation following JunD knockdown including PRDX3, PEA15, KIF2C and CDK2.

In the present disclosure, JunD regulated genes which are involved in cell cycle regulation were identified. A relationship at the molecular level between these two components, JunD and its target genes, is disclosed which establishes the mechanism (s) of JunD-driven cell proliferation in prostate cancer cells. Using an integrative genomic and proteomic approach, 115 common target genes and proteins were identified that were significantly downregulated in JunD-deficient cells compared to the control cells.

In addition, pathway analysis by Ingenuity Pathway Analysis (IPA) was implemented to identify alterations in signaling pathways in JunD-deficient prostate cancer cells. These analyses suggest that 75 out of the 115 identified molecules were involved in cell cycle control/regulation and their expression is dependent on the presence of JunD. The majority of these genes/proteins were associated with MYC signaling pathway, a known master regulator of the cell cycle and key player in carcinogenesis, whose expression was also inhibited after JunD KD suggesting JunD regulation of target genes via MYC pathway in prostate cancer cells. Experimental data establishes the interplay between JunD and c-MYC transcriptional regulation of genes leading to prostate cancer development and progression.

Studies show that upon stimulation, JUN family members exhibit a rapid upregulation that effectively stimulates transcription of genes important for entry into the G1 and S phases of the cell cycle such as the cyclins including cyclin D1, cyclin A, and cyclin E [55, 57, 59]. Likewise, the inhibition of several AP-1 family members depending on the context of a cell results in decrease in cyclins expression and cell growth inhibition [35, 57, 60]. The present disclosure confirms that JunD downregulation results in a significant decrease in many key players involved in cell cycle regulation including cyclin-dependent kinases (CDKs)-CDK1, CDK2, and CDK4.

Contrary to previous findings which indicate c-MYC as the regulator of cell cycle/proliferation, reports suggest JunD as the key player in cell proliferation, cell cycle regulation, and regulating several genes involved in cell cycle control including c-MYC [29].

AP-1 proteins are also known to be involved in transformation and have been associated with aggressive clinical outcome in prostate cancer [33, 35]. AP-1 family members can form complexes with a variety of TFs to carry out biological functions. Researchers previously demonstrated that JunD in prostate cancer cell migration differs from c-JUN and JunB because of specific AP-1 target genes with neighboring ETS and AP-1 binding sites in cis-regulatory elements that allows for specificity mechanisms [20].

Experimental data confirms previous findings that among other cell cycle related genes, PRDX3, a known c-MYC target gene [38] is also an identified JunD target gene that is required for cell proliferation of prostate cancer cells. Furthermore, the MYC signaling pathway was implicated as the upstream regulator of these genes, indicating c-MYC's control on some of JunD target genes. The present disclosure confirmed JunD regulation of these proteins. Experimental data further demonstrated that in JunD-deficient cells that c-MYC, among other required proteins of cell cycle regulation including PRDX3 and CDK2, exhibited a decrease in its protein levels, while the overexpression of JunD enhanced their protein levels thereby supporting the increase of cell proliferation in prostate cancer cells.

As disclosed herein, in contrast to DU145 cells, JunD knock-down (KD) resulted in the reduction of c-MYC protein levels in PC3 cells, but not its mRNA levels, suggesting posttranscriptional regulation of c-MYC by JunD. Whereas in DU145 cells, JunD KD caused a significant decrease in both mRNA and protein levels suggesting that c-MYC may also have AP-1 binding sites present in its promoter regions in this specific prostate cancer cell line. MYC can be activated by multiple mechanisms in cancer cells including transcriptional regulation, mRNA stabilization, and protein overexpression and stabilization [79]. Based on experimental data, JunD target genes down-regulated in JunD deficient cells were identified to be involved in some of the top mechanistic networks (according to IPA) including RNA processing (DIMT1, EFTUD2, HTT, PNN, RPS6/7, PTBP1, RRP1B, and SNRPD1), translational control (EIF1, EIF2AK2, ILF3, RPS5, and ANAX2), and protein stability (HSPA8, DNAJA1, USP15, XIAP, NEDD4, and ANAPC2).

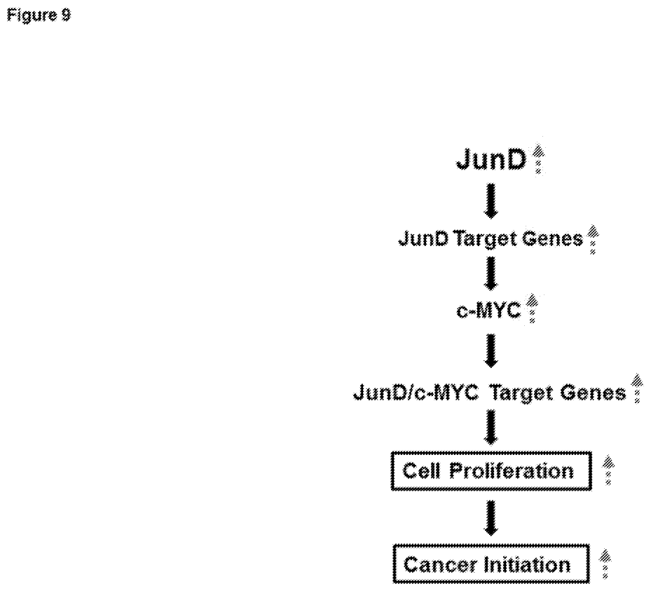

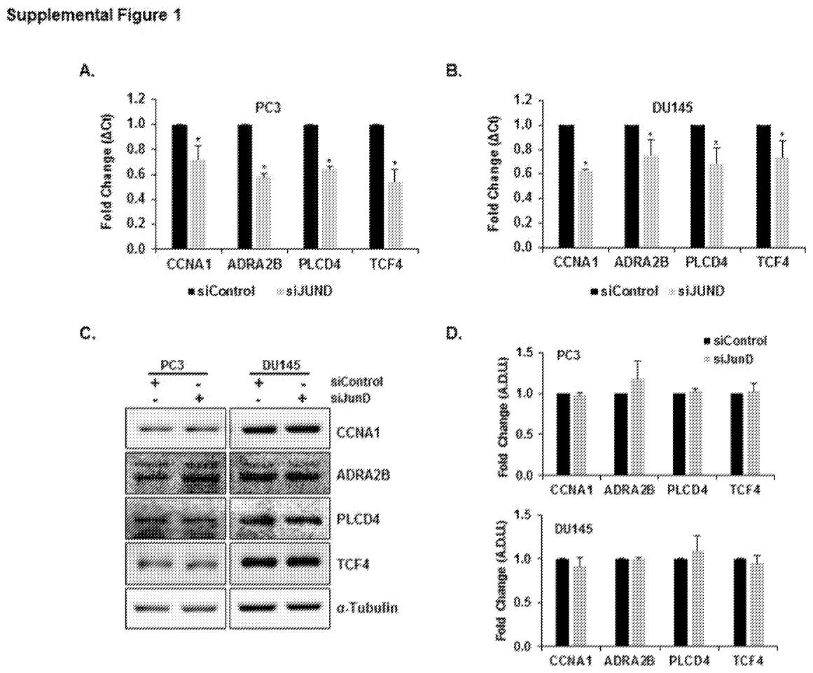

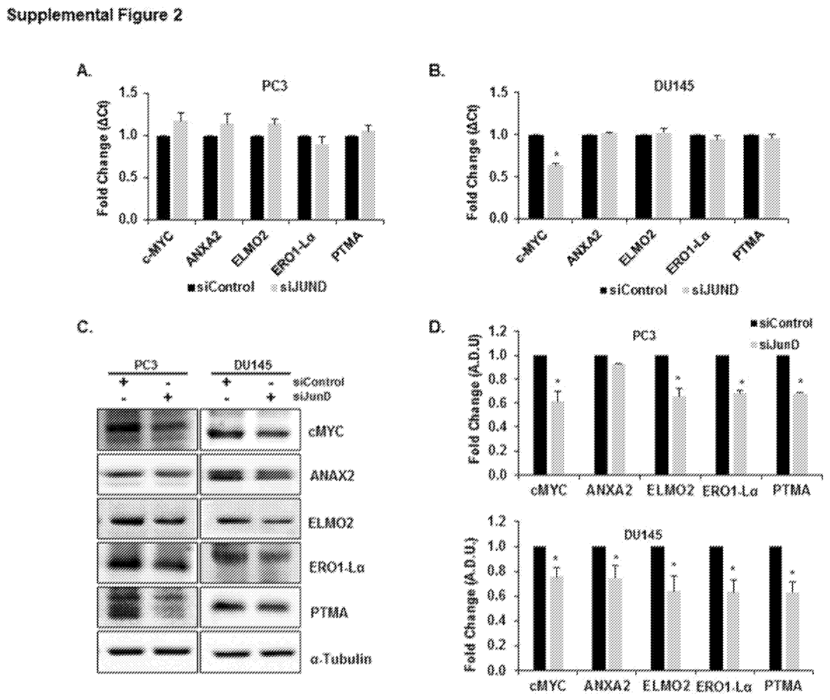

The present disclosure investigates the question is how JunD mediates its effects on c-MYC protein to carry out cell proliferation and ultimately support prostate cancer progression. FIG. 9 outlines a schematic in which the overexpression of JunD directly activates its target genes, whose products target c-MYC, thereby leading to an increase in c-MYC protein levels which then leads to the activation of downstream targets (JunD/c-MYC target genes). This cascade of events leads to the increase in cell proliferation of prostate cancer cells and the initiation of carcinogenesis (FIG. 9).

Taken together, these results show that JunD is essential for prostate cancer cell proliferation, required for the expression of cell cycle-related genes, and that it acts upstream of c-MYC which is currently recognized as a major factor in the initiation of prostate carcinogenesis. Although the majority of research reports related to c-MYC focused primarily on its normal physiological functions as well as its overexpression leading to carcinogenesis, the present disclosure elucidates mechanisms involved in upstream regulation of c-MYC.

The enclosed data identified JunD target genes that may be required for the upregulation of c-MYC protein levels as well as genes that function downstream c-MYC, such as PRDX3, to promote prostate cancer. Identification of JunD target genes followed by the development of approaches to inhibit their expression and/or function will lead to the development of therapeutic and chemo-preventive strategies to interfere with deregulation of cell proliferation and early stages of carcinogenesis.

Microarray and Proteomics data were deposited into the GEO database. The accession number for the super series, which contains both microarray and proteomics datasets is GSE118123 (available at https://www.ncbi.nlm.nih.gov/geo/info/linking.html).

### EXPERIMENTAL

### Materials and Methods

### Chemical and Reagents

Antibodies against JunD (Cat. #sc-74), PRDX3 (Cat. #sc-59663), and c-MYC (Cat. #sc-40) were purchased from Santa Cruz Biotechnology, Inc. (Dallas, Tex.). Antibodies against CDK2 (Cat. #sc-2848), CDK4 (Cat. #sc-166373), KIF2C (Cat. #sc-81305), EIF1/B (Cat. #sc-390122), PEA15 (Cat. #sc-166678), Cyclin A or CCNA1 (Cat. #sc-271682), α_{2B}-AR or ADRA2B (Cat. #sc-390430), PLCD4 (Cat. #sc-373875), TCF4 (Cat. #sc-166699), Annexin II or ANAX2 (Cat. #sc-28385), ELMO2 (Cat. #sc-365739), ERO1-Lα (Cat. #sc-100805), and Tropomyosin or PTMA (Cat. #sc-74480) were all provided as samples from Santa Cruz Biotechnology, Inc. (Dallas, Tex.). The antibody against anti-α-Tubulin (Cat. #T5168) was purchased from Sigma-Aldrich (St. Louis, Mo.). Anti-mouse IgG-HRP and Goat anti-rabbit IgG-HRP (immunoglobulin horseradish peroxidase) were purchased from GE Healthcare (Piscataway, N.J.) and Promega (Madison, Wis.), respectively. Small interfering RNA transfection reagent (Cat #sc-29528), JunD (Cat. #sc-3578), PRDX3 (Cat. #sc-40833), and Control-A (Cat. #37007) siRNAs were all purchased from Santa Cruz Biotechnology, Inc. Lipofectamine 3000 Transfection Reagent and DAPI were purchased from ThermoFisher Scientific, Inc. (Waltham, Mass.). JQ1 inhibitor (Cat #27400) was purchased from BPS Bioscience (San Diego, Calif.) and dissolved in dimethyl sulfoxide (Fisher Scientific) to a stock concentration of 10 mM, aliquoted and stored at −80° C. G418 (Cat. #345810) was purchased from Calbiochem.

### Cell Lines and Cell Culture

Human prostate cancer cell lines (PC3 and DU145) were purchased from ATCC (Manassas, Va.). Cells were cultured in the recommended growth media [MEM media supplemented with 5% fetal bovine serum (FBS)] in 100% humidity at 37° C. with 5% CO₂ as described previously [29]. DU145 cells overexpressing JunD (D1), generated from a previous study were cultured as above, with an addition of 200 ng/ml G418 [29].

### Generation of JunD Knock-Out (KO) Cells by CRISPR/Cas9 Genome Editing

PC3 JunD knock-out cells were generated using CRISR/Cas9 as previously described [44, 45]. In brief, CRISPR/Cas9 single-guide RNAs (sgRNAs) targeting 2 locations on JunD exon 1 were identified using the CRISPR design tool provided by Zhang's Lab at MIT (available at http://crispr.mit.edu/) as follows, 5′-GCCTACCCCCCTGCGCGCCGA-3′ and 5′-GTTCGCGTAGACAGGCGCTTC-3′. Each sgRNAs were cloned into an all in one-WT Cas 9″ plasmid vector, previously generated in Dr. Chunliang Li's Lab, St. Jude Children's Research Hospital, Memphis Tenn. Plasmids containing sgRNAs were validated by Sanger sequencing using the U6 promoter forward primer 5′-GAGGGCCTATITCCCATGAT-3′ and then transfected into PC3 cells using Lipofectamine 3000 reagent, according to the manufacture's protocol. Western blot analyses were performed to confirm the knock-out of JunD protein. JunD knock-out cells were used in additional functional assays.

Transient Transfection with JunD, PRDX3, and Control siRNA

The transient knock-down of JunD or PRDX3 protein in PC3 and DU145 cells were performed using previously described methods [29]. In brief, PC3 and DU145 cells were transfected with 60 nM of JunD, PRDX3, or control siRNA using transfection reagents (Santa Cruz Biotechnology) following manufacturer's recommendations. Seventy-two hours after transfection, the knock-down of JunD or PRDX3 expression in PC3 and DU145 were confirmed by Western blot analysis and then subjected to several functional analyses.

### Cell Proliferation Assays

Cell growth assays were performed to examine the growth rate of PC3 wt and PC3 JunD knock-out cells generated by CRISPR/Cas9. Cells were seeded in a 6-well plate at a density of 1×10⁵ cells/well and the growth rate was determined after 4 days. Cells were then trypsinized and counted using a cellometer as previously described [29, 46]. Cells were also seeded in 24-well plate at a density of 2×10⁴ cells/well and counted on days 1, 2, 4, 6, and 8 and counted using a hemacytometer. For proliferation assays of transfected cells, cells were seeded in a 6-well plate at a density of 1.5×10⁵ cells/well. Cell proliferation was examined 72 hrs following transient transfection with JunD or PRDX3, and control siRNAs. Cell growth assays were performed using cell counting.

Treatment of Cells with JQ1 Inhibitor

DU145 vector and DU145 JunD overexpressed (D1) cells plated at a density of 2×10⁴ cells/ml in 24 well plates (for cell proliferation assays) or at a density of 1.5×10⁵ cell/ml in 6 well plates (for cell lysates) were treated in the presence or absence of 5 μM JQ1 for 72 hrs. Control cells were treated with DMSO. Cells were then counted using a hemacytometer and cell lysates were collected for western blot analyses.

### Immunofluorescence of F-Actin Staining

PC3 wt and PC3 KO cells generated by CRISPR/cas9 were grown (0.5×10⁵) on glass cover slips for 72 hrs, fixed and permerabilized as previously described [44]. In brief, cells were stained with rhodamine-phalloidin for 30 min and DAPI for 10 min to detect F-actin filaments and the nuclei, respectively. Slides were mounted in Vectashield mounting medium (Vector Laboratories, Burlingame, Calif.) and images captured using Carl Zeiss 200M inverted microscope (Carl Zeiss, Thornwood, N.Y.) at 20× magnification [29]. The cell area was determined using Image J software.

### Total RNA Preparation

Total RNA from all human prostate cells/cell lines used in this study was isolated using TRIzol (Life Technologies, Grand Island, N.Y.) as previously described [29, 46, 47]. For quantitative real-time PCR (qRT-PCR), RNA concentration and purity was determined using a Nanodrop 2000c Spectrophotometer (A260/280 ratio≥1.9) as previously described [46]. For subsequent microarray studies, RNA samples (triplicate samples from each condition) were sent to Georgia Institute of Technology (Georgia-Tech, Atlanta, Ga.) for processing and analyses. The quality of the RNA was verified using an Agilent Bioanalyzer (Agilent Technologies) [48]. RNA samples with RNA integrity number (RIN) of 10 were used for microarray analysis. The samples were diluted to a final concentration of 500 ng/μl and applied to an RNA chip according to the manufacturer's instructions. GeneChip Human Genome U133 Plus 2.0 Arrays (Affymetrix) was used following manufacturer's recommendations [48, 49].

Quantitative Real-Time PCR (qRT-PCR) Analysis

The synthesis of cDNA from total RNA (2 μg) by reverse transcription was performed as previously described [50]. qRT-PCR was carried out in triplicate using GoTaq Master Mix (Promega, Madison, Wis.) on BioRad CFX Connect Real time PCR System (BioRad, Hercules, Calif.) as previously described [46]. Melting curves were generated to confirm the amplification of a single PCR product. Quantitation of the PCR results were based on the threshold cycle (Ct) and normalized to human GAPDH as previously described [46]. All human PCR primers were designed using Primer3 Plus software (available at https://primer3plus.com/cgi-bin/dev/primer3plus.cgi) and synthesized by Integrated DNA Technologies (Coralville, Iowa). All primers with their respective sequences are listed in Supplemental Table 1. Independent experiments were repeated at least three times for each sample/condition.

### Microarray Analysis

To investigate differential gene expression after JunD knockdown by siRNA in PC3 cells, microarray analyses were conducted on the GeneChip® Human Genome U133 Plus 2.0 Arrays (Affymetrix), providing comprehensive analysis of genome-wide expression on a single array. Gene expression signals from each array were processed to Affymetrix. CEL files using the Affymetrix Expression Console (EC) Software Version 1.4 using the Robust Multi-Array Average (RMA) normalization method [48, 51]. The normalized expression values from all samples (N=2, in triplicates) were log 2 transformed. Differentially expressed genes were identified as fold changes (FC)>1.1 (up or down) and p-value<0.05. Genes were annotated using Ingenuity Pathway software (available at http://www.ingenuity.com/) for cellular functions during differentiation and interactions and were mapped according to their instructions [52].

### Western Blot Analysis

Following siRNA transient transfections, PC3 and DU145 cell lysates from several independent experiments were collected as previously described [29]. In brief, equal amounts of protein (50 μg) were separated by electrophoresis using 10% SDS-polyacrylamide gels and then transferred to PVDF membranes (Millipore, Billerica, Mass.). After being blocked with 5% milk, the membranes were incubated with specific primary antibodies (1:800 dilution for JunD; 1:500 dilution for anti-PRDX3, anti-CDK2, anti-CDK4, anti-KIF2C, anti-EIF1, anti-PEA15, anti-CCNA1, anti-ADRA2B, anti-TCF4, anti-ANAX2, anti-ELMO2, and anti-ERO1-La; 1:200 dilution for anti-PLCD4, anti-c-MYC, and anti-PTMA; 1:3000 dilution for anti-α-Tubulin) overnight at 4° C. and then incubated with appropriate horseradish peroxidase-conjugated secondary antibody for 1 h. The blots were developed using Millipore Luminata Forte (EMD Millipore, Billerica, Mass.) and visualized by Syngene PXI 6 imagining system (Syngene, Frederick, Md.). All blots were probed for α-Tubulin and used as loading controls. The relative intensities of specific protein were determined by ImageJ software (NIH version: 1.8.0_112).

### Proteomics

Cell Lysis and Protein Extraction.

PC3 cell were subjected to proteomic analysis as previously described, with minor modifications [53, 54] In brief, PC3 cells were plated at a total density of 1×10⁶ cells in a 6-well plate per condition and transfected the next day with control or JunD siRNA for 72 h. Biological replicates were prepared using the same conditions. Following siRNA transfections, the cell pellets from PC3 control and PC3-JunD KD cells were lysed with 1 ml M-Per Mammalian Protein Extraction Reagent (Thermo Scientific) containing 10 μl phosphatase inhibitor and 10 μl protease inhibitor (Thermo Scientific) according to manufacturer's instructions. The samples were sonicated followed by centrifugation at 16,000×g for 5 min. The supernatant of each sample was collected and the protein concentrations were determined by the BCA protein assay (Pierce Biotechnology, Rockford, Ill.) following manufacturer instructions. Samples were subjected to reduction, alkylation, and trypsin digestion as previously described [53, 54]. The trypsin-digested samples were used in the next procedure.

Isobaric Labeling with Tandem Mass Tag (TMT).

Tandem mass tags (TMT⁶) (Thermo Scientific) with increasing molecular weights ranging from 126-131 Da were applied as isobaric tags to determine differential protein levels between PC3 control cells and PC3 JunD knockdown cells as previously described [53, 54]. The labeled peptide mixtures were combined at equal ratios and purified by a strong cation exchange (SCX) column.

Fractionation of Labeled Peptide Mixture by Using a Strong Cation Exchange Column.

The combined TMT-labeled peptide mixtures were fractionated with a SCX column (Thermo Scientific) on a Shimadzu Ultra-Fast Liquid Chromatography (UFLC) equipped with an ultraviolet detector (Shimadzu, Columbia, Md.), and a mobile phase consisting of buffer A (5 mM KH2PO4, 25% acetonitrile, and pH 2.8) and buffer B (buffer A plus 350 mM KCl), as previously described [53, 54]. In total, sixty fractions were collected, lyophilized, and combined into 14 final fractions based on SCX chromatogram peaks. The collected fractions were desalted using a C18 solid-phase extraction (SPE) column (Hyper-Sep SPE Columns, Thermo Scientific) as previously described [53, 54]. Briefly, the 14 combined fractions were each adjusted to a final volume of 1 ml containing 0.25% trifluoroacetic acid (TFA) solution. The eluted samples were lyophilized prior to the liquid chromatography mass spectrometry to liquid (LC-MS/MS) analysis.

LC-MS/MS Analysis on LTQ-Orbitrap.

Peptides were analyzed on an LTQ Orbitrap.XL (Thermo Scientific, Waltham, Mass.) instrument interfaced with an Ultimate 3000 Dionex LC system (Dionex, Sunnyvale, Calif.). High mass resolution was used for peptide identification and high energy collision dissociation (HCD) was employed for reporter ion quantification as previously described [53, 54].

Database Search and TMT Quantification.

The protein search algorithm SEQUEST was used to identify and quantify unique peptides using the Proteome Discoverer data processing software (version 1.2, Thermo Fisher Scientific, Waltham, Mass.). Peptides reported by the search engine were accepted only if they met the false discovery rate of P<0.05 (target decoy database). The ratios of TMT reporter ion abundances in MS/MS spectra generated by HCD from raw data sets were used for TMT quantification. Fold changes in proteins between PC3 control and PC3-JunD KD samples were calculated as previously described [53, 54].

Ingenuity Pathway Analysis (IPA).

Data sets generated from microarray and proteomic mass spectrometric analyses were analyzed using Ingenuity Pathway Analysis (IPA) software (Ingenuity Systems, Inc., Redwood City, Calif.; available at http://www.ingenuity.com) [52]. Ingenuity Knowledge Based tool was used to identify all significant biological functions and canonical pathways that involve differentially expressed genes (DEGs) and differentially regulated proteins (DEPs). The IPA program applies Fisher's exact test to calculate a p-value that represents the probability of the DEGs and DEPs in the pathway being found together due to random chance. Specifically, genes and proteins identified in the microarray and proteomics, respectively, with differential expression p-values<0.05 and fold-changes≥1.5 were used as focus genes/proteins. Pathways with p-values<0.05 were considered significantly enriched.

Analysis of JunD Levels in Prostate Tissues from TCGA.

The expression matrix of JunD in prostate tissues was obtained from the gene expression of RNAseq (Illumina HiSeq) dataset of GDC Prostate Cancer cohort in the TCGA database. The raw data of gene expression levels were log 2(x+1) transformed and processed at the UCSC Xena repository as previously described [55]. Reprocessed data were downloaded using UCSC Xena Functional Genomics Explorer (available at https://xenabrowser.net/).

Statistical Analysis.

All data are presented as the mean±standard deviation (SD). Statistical analyses were performed using one-way ANOVA. P value<0.5 was considered statistically significant.

### Results

### JunD KO Attenuates Cell Proliferation of Prostate Cancer Cells

JunD plays an essential role in cell proliferation of prostate cancer cells [29]. To confirm JunD's role in cell proliferation, JunD knock out (KO) prostate cancer cells (PC3-JunD sg1/2-1) using CRISPR Cas 9 genomic editing were generated (FIG. 1). A complete knockout of JunD protein in Clone sg1/2-1 was demonstrated (insert, FIG. 1). In the absence of JunD protein, cell proliferation is significantly reduced (61%, p<0.001) compared to PC3 wt (control) cells (FIG. 1A) with a significant decrease in proliferation in a time-dependent manner (FIG. 1B) and a decrease in cell size (FIG. 1C). Because these JunD KO cells' growth rate slowed down tremendously causing difficulty in carrying out additional functional studies, cells with JunD transiently knockdown were utilized throughout the remainder of the study.

### JunD Knockdown Decreases Expression of Cell Cycle-Related Genes Including MYC

JunD knock-down (KD) results in cell cycle arrest in G1-phase concomitant with a decrease in the levels of cyclin D1, Ki67, c-MYC, and Id1, but an increase in p21 protein levels [29]. Furthermore, the over-expression of JunD significantly increased cell proliferation in these cells suggesting that JunD regulates the expression of genes which are required for the progression of cell cycle [29]. To test this possibility and to elucidate the key molecules and/or signaling pathways in JunD-mediated cell proliferation of prostate cancer cells, PC3 cells treated with siRNA to knock-down endogenous JunD and PC3 control cells were subjected to microarray and proteomic analyses as illustrated in FIG. 2. An 85% knock-down of JunD protein was confirmed in comparison with cells transfected with control siRNA as determined by Western blot analysis (FIG. 2). Following microarray and proteomic analysis, differentially expressed genes (DEGs) and proteins (DEPs) that were down-regulated or up-regulated in PC3 JunD KD cells were identified compared to siControl PC3 cells. From microarray analysis, a total of 54,675 molecules were quantified, and among these, 3,103 were found to have differential expression (p<0.05), including 1,598 upregulated and 1,505 downregulated molecules (Table in FIG. 2). From proteomic analysis, a total of 5,605 protein molecules were quantified, and among these, 2,056 were found to have differential expression (p<0.05), including 1,007 upregulated and 1,049 downregulated protein molecules (Table in FIG. 2).

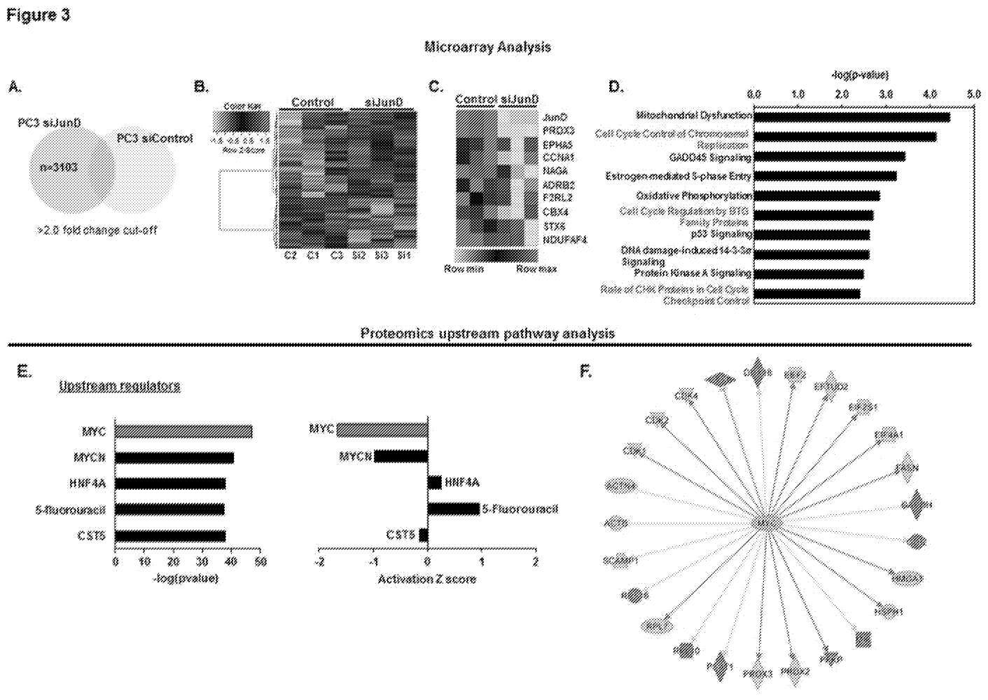

A signature was detected by recruiting several probes with a cutoff value of >2.0-fold change in JunD-deficient PC3 cells compared with control cells as depicted in the Venn diagram (FIG. 3A). Hierarchical clustering analysis was used to compare differential (p<0.01) JunD expression between PC3 control and PC3-JunD KD cells as shown in (FIG. 3B). The top 20 up- or down-regulated genes are shown in Supplemental Table 2. As a first step, genes that were down-regulated as a result of JunD KD were studied [29]. The top ten down-regulated genes from microarray data are shown in a heat map visualization and include JunD, PRDX3, EPHA5, CCNA1, NAGA, ADRB2, F2RL2, CBX4, STX6, and NDUFAF4 (FIG. 3C).

To identify altered pathways and to explore the potential function(s) of down-regulated mRNAs in JunD-deficient cells compared to control cells, Ingenuity Pathway Analysis (IPA) was used to identify the biological processes, molecular and cellular functions, and their possible involvement in diseases and disorders. The pathways and functions were determined by an enrichment score (p<0.05) as previously described [52]. The data show that in comparison with siControl PC3 cells, JunD deficiency was associated with several changes in specific signaling pathways. Cell cycle control/regulation was predicted to be one of the top pathways whose members exhibited decreased gene expression following JunD knock-down (FIG. 3D). Similar to transcriptome analysis, most significant differences in protein expression levels (p<0.05) were also observed in proteins involved in cell cycle regulation (Top 20 DEPs up or down (p<0.05), Supplemental Table 3). Furthermore, in an upstream pathway analysis using proteomics data, MYC, a key regulator of cell cycle control, was one of the top upstream regulators (FIG. 3E, left) in which its activity was dramatically inhibited following JunD depletion (FIG. 3E, right). IPA analysis of proteomics data focusing on MYC and its interaction with target molecules are illustrated in FIG. 3F.

### Integrated Analysis of PC3-JunD Knockdown Cells Transcriptome and Proteome

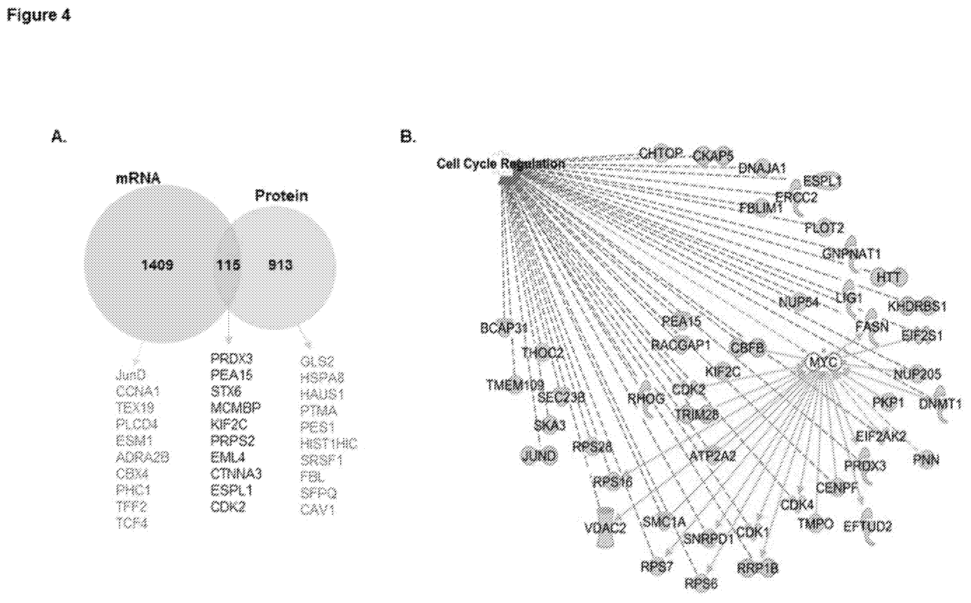

To further determine the molecular signature associated with JunD-mediated cell proliferation/cell cycle regulation, a comparative analysis of genomic and proteomic data was performed using IPA (using only significantly down-regulated molecules, p<0.05) and investigated the functional annotation and interrelation of 3 groups: unique genes found only in microarray data, unique proteins found only in proteomic data, and genes/proteins shared between both microarray and proteomic data. The overlap between protein and mRNA analyses are depicted in FIG. 4A. The top 10 down-regulated molecules for each category are listed below its respective group. This data analysis revealed that 92% of genes (out of 1,524 genes) and 88% of proteins (out of 1,028 proteins) were unique to microarray or proteomic data, respectively, in JunD-deficient cells, which are 1409 DEGs and 913 DEPs that were down-regulated. Additionally, 45% genes/proteins (115 molecules) were shared between the two groups. Using canonical pathway analysis, these molecules were categorized to related biological pathways. Among other significantly altered signaling pathways, cell cycle control of chromosomal replication was identified as one of the top significantly enriched canonical pathways of the genes/proteins altered in JunD deficient cells (Fisher' exact test, P<10⁻³, Table 1, Top Canonical Pathways section) in which 75 of these molecules were identified as involved in cell cycle regulation (top 10 listed in FIG. 4A, full list provided in Supplemental Table 4). IPA analysis also predicted that the majority of these molecules are associated with the MYC pathway, which indicates their possible role in cancer progression in addition to their involvement in cell cycle regulation (FIG. 4B). Furthermore, the annotation of these molecules indicate cancer among other diseases as the top 5 most associated disease and function based on the ranking of −log 10P (Table 1, Top Diseases section). These analyses also highlighted JunD target molecules that participated in major molecular functions including RNA Post-Transcriptional Modification (63%, p-value=2.10E-04-3.8E-11), Cell Death and Survival (59%, p-value=7.01E-03-4.77E-10), Cell Cycle 41%, p-value=7.01E-03-6.80E-09), Protein Synthesis (24%, p-value=8.29E-04-1.72E-07), and Cellular Assembly and Organization (45%, p-value=7.01E-03-2.35E-07), as shown in Supplemental Table 5.

### Validation of JunD Target Genes and Proteins Identified by Microarray and Proteomics

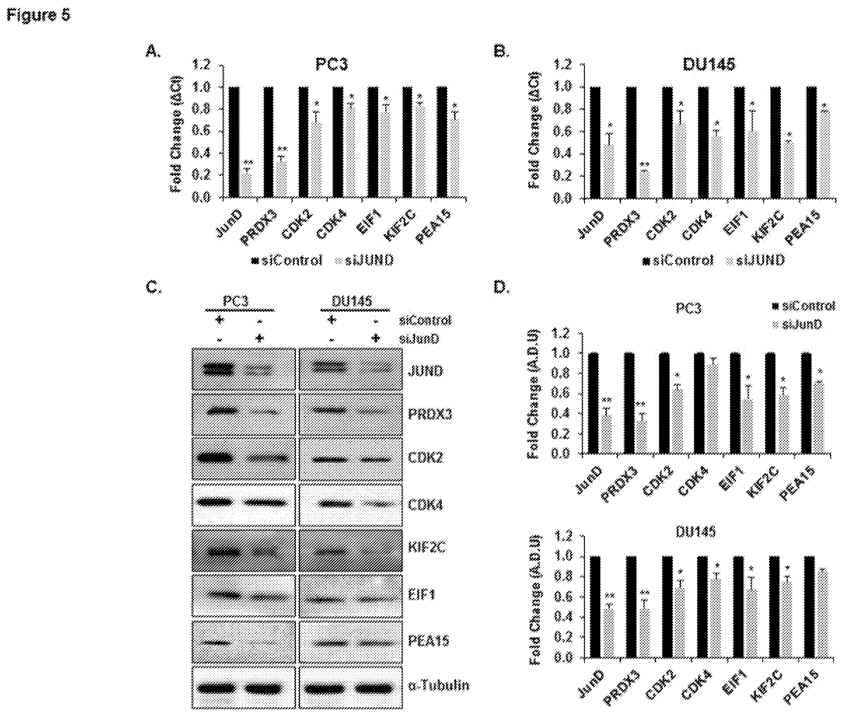

Among the 75 down-regulated genes/proteins identified to play a role in cell cycle regulation, six genes/proteins (PRDX3, CDK2, CDK4, EIF1, KIF2C, and PEA15) which were significantly down-regulated in PC3-JunD KD cells vs PC3 control cells were the primary focus of study. Validation of microarray data was carried out using qRT-PCR on the same RNA samples used for transcriptome microarray analysis. Western blot analyses were performed to detect and validate the expression levels of these proteins. Additional independent experimental sample preparations were also collected for both RNA and protein for additional biological replicates. The selected gene/protein expression levels in DU145 cells after the knock-down of endogenous JunD was also confirmed. As shown in FIG. 5, the expression patterns of the 6 selected significantly down-regulated gene/proteins in qRT-PCR (FIG. 5A, B) and Western blot analysis (FIG. 5C) were consistent with the microarray and proteomic analysis (P≤0.05), which demonstrated the reliability of the microarray and proteomic data. Among the 6 validated gene/proteins, the most significantly down-regulated gene/protein was PRDX3 (p<0.01) in both PC3 and DU145 JunD-deficient cells. JunD mRNA and protein levels were also determined by qPCR and western blot analysis which confirmed a significant decrease of JunD protein in PC3 (62% decrease, p<0.05) and DU145 cells (52% decrease, p<0.05) in comparison with the cells transfected with the control siRNA. The relative protein levels of the selected genes normalized with α-Tubulin levels are indicated adjacent to the Western blot image (FIG. 5D). These results confirmed that the selected genes are indeed JunD target genes. Gene expression and protein levels of molecules that exhibited a significant decrease in mRNA levels, but not protein levels were also confirmed (Supplemental FIG. 1) and also molecules which exhibited a significant decrease in protein levels, but not in mRNA levels (Supplemental FIG. 2).

JQ1, a c-MYC Inhibitor, Suppresses JunD-Mediated Cell Proliferation of Prostate Cancer Cells

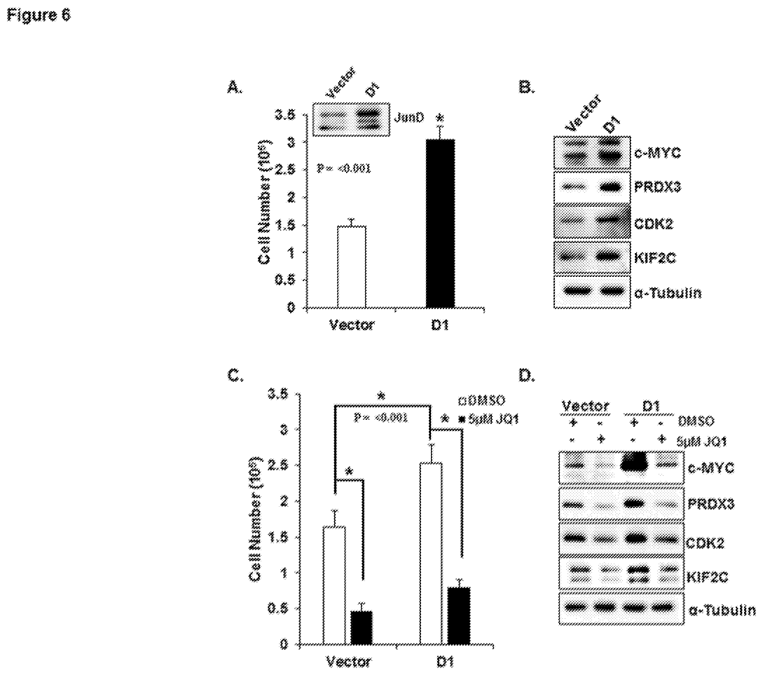

On the basis of the findings that JunD-deficient cells exhibited a decrease in the expression of JunD target genes that are involved in cell cycle regulation, the role of JunD was further examined with respect to their protein levels in DU145 cells over-expressing JunD. The over-expression of JunD protein and the increase in proliferation of JunD over-expressing cell line (D1) compared to the control (vector) cells was confirmed (FIG. 6). Cells were plated at an initial density of 1×10⁵, followed by cell counting after 72 hrs. As shown in FIG. 6A, D1 cells exhibited a 2-fold increase±0.27 (p<0.05) in growth rate compared to the control cells, which also correlated with the increase in JunD protein levels (inset). D1 cells also displayed a significant increase in c-MYC, PRDX3, KIF2C, and CDK2 protein levels compared to the control cells (FIG. 6B). To further demonstrate that JunD target genes require MYC signaling even in the presence of JunD, both control and D1 cells were treated with 5 μM JQ1, a c-MYC inhibitor [56] for 72 hrs. JQ1 significantly reduced (FIG. 6C) cell proliferation (p<0.001) and decreased JunD target genes protein levels in both vector and D1 cells (FIG. 6D).

### PRDX3 Knock-Down Inhibits Cell Proliferation in Prostate Cancer Cells

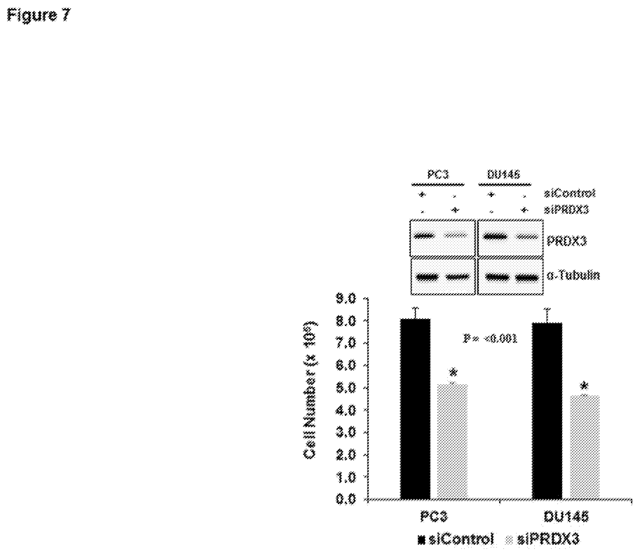

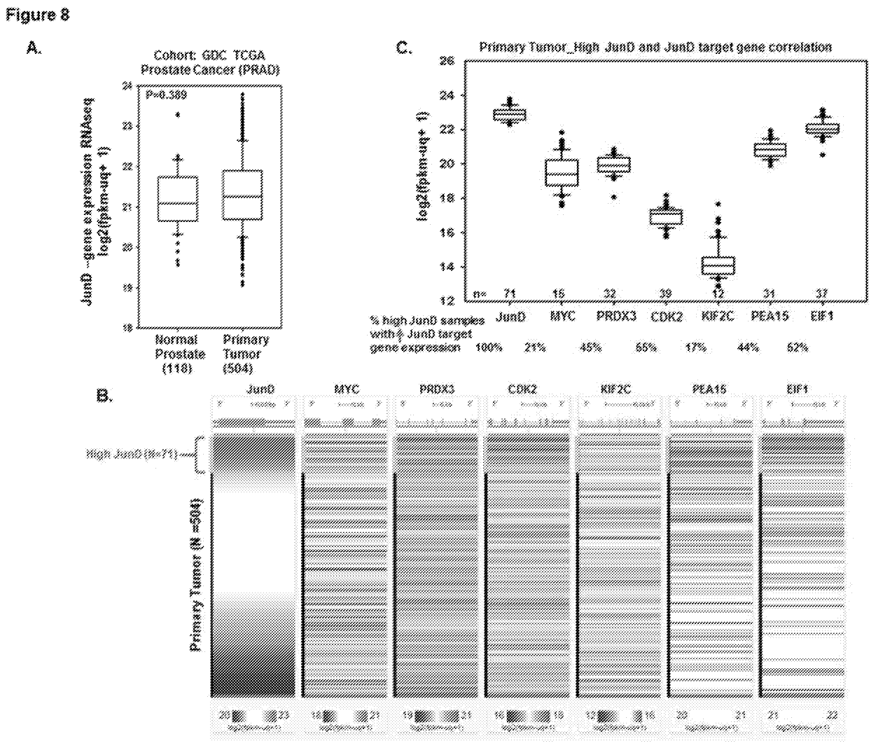

To confirm JunD target genes requirement for prostate cancer cell proliferation, PRDX3, the top hit that was downregulated by JunD KD in PC3 cells in both groups, was examined. PRDX3 is also a known key player in cell proliferation and is involved in promoting cell survival in prostate cancer [57]. PRDX3 KD by siRNA in PC3 and DU145 cells was confirmed by western blot analysis, while the control siRNA had no effect on its protein levels (inset, FIG. 7). PRDX3 protein levels were significantly reduced (50% decrease, p<0.05) in comparison to the controls in both PC3 and DU145 cells. The relative protein levels of PRDX3 were normalized to α-Tubulin (quantitative data not shown). Proliferation of PC3-PRDX3 and DU145-PRDX3 cells were also examined by cell counting 72 hrs after siRNA treatment. The data showed that PC3 and DU145 PRDX3-deficient cells exhibited a significant reduction in cell proliferation ((PC3, 36% inhibition, p<0.05) and (DU145, 42% inhibition, p<0.05)) cells (FIG. 7). These results suggest that JunD target gene, PRDX3, is required for cell proliferation of prostate cancer cells.

### JunD Expression in Prostate Tissue

The expression profile of JunD in prostate tissues using The Cancer Genome Atlas (TCGA) dataset was explored. There were a total of 623 samples, including 504 prostate primary tumor tissues, 118 normal prostate tissues, and one metastatic tissue. Compared to normal prostate tissues, there was no significant difference in JunD levels in the prostate primary tumor tissues (FIG. 8A). Although there was no significant difference between these two groups, JunD levels were examined only in primary tumor samples. The results show that 71 primary tumor tissues had high JunD levels (FIG. 8B). c-MYC, PRDX3, CDK2, KIF2C, PEA15, and EIF1 levels were examined to see if there was a correlation with high JunD levels in primary tumor samples as present data showed in the prostate cancer cell lines under study (FIG. 8B). The results showed that in the 71 primary tumor samples with high that JunD, there were 21% with high c-MYC, 45% PRDX3, 55% CDK2, 17% KIF2C, 44% PEA15, and 52% EIF1, as shown in FIG. 8C.

TABLE 1
Summary of Ingenuity Pathway Analysis.

| Pathway Analysis | P-value |
|---|---|
| Top Canonical Pathways | |
| EIF2 Signaling | 5.30E−09 |
| Regulation of eiF4 and p70S6K Signaling | 1.32E−06 |
| Cell Cycle Control of Chromosomal Replication | 1.55E−06 |
| Top Upstream Regulators | |
| MYC | 1.13E−10 |
| E2F1 | 1.71E−09 |
| sirolimus | 1.76E−06 |
| Top Molecular and Cellular Functions | |
| RNA Post-Transcriptional Modification | 2.10E−04-3.84E−11 |
| Cell Death and Survival | 7.01E−03-4.77E−10 |
| Cell Cycle | 7.01E−03-6.80E−9 |
| Protein Synthesis | 8.29E−04-1.72E−07 |
| Top Diseases | |
| Cancer | 7.01E−03-2.31E−08 |
| Organismal Injury and Abnormalities | 7.01E−03-2.31E−08 |
| Tumor Morphology | 6.35E−03-2.31E−08 |
| Top Networks | |
| Cell Morphology, Cellular Function and | |
| Maintenance | |
| Cell Cycle, Cell Death and Survival, Cellular | |
| Assembly and Organization | |
| Protein Synthesis, RNA Post-Transcriptional | |
| Modification | |

The includes shared analysis between Microarray and Proteomics ± JUND of down- regulated (p < 0.05) molecules.

SUPPLEMENTAL TABLE 1
Human Primers used for qRT-PCR assays.

| Gene | Forward | Reverse |
|---|---|---|
| Symbol | 5′ → 3′ | 5′ → 3′ |
| ADRA2B | GCTGTGGTCA | GCGGAAGTCC |
| | TTGGCGTTTT | TGGTTGAAGA |
| ANXA2 | AATCCTGTGC | TGCTGCGGTT |
| | AAGCTCAGCT | GGTCAAAATG |
| CCNA1 | GGTCCCGATG | CTTTCCAGCT |
| | CTTGTCAGAT | GGAGGGAAGG |
| CDK2 | CATTCCTCTT | CAGGGACTCC |
| | CCCCTCATCA | AAAAGCTCTG |
| CDK4 | AGCTCCCGAA | CATCTCGAGG |
| | GTTCTTCTGC | CCAGTCATCC |
| EIF1 | TGTCCAAGGG | CAGCTGATCG |
| | ATCGCTGATG | TCCTTAGCCA |
| ELMO2 | TTTGCCCTCC | CAAGGATCTC |
| | AAACCCAACT | GGGCTGGATC |
| ERO1Lα | TGAAGAGGCC | TCCACTGCTC |
| | GTGTCCTTTC | CAAGTCGTTC |
| JunD | TGGAAACACC | TCAGCGCGTC |
| | CTTCTACGGC | CTTCTTCATC |
| KIF2C | CACTCGCATG | CATCTCCTCG |
| | TCCACTGTCT | CTGACCATCC |
| c-MYC | CGTCCTCGGA | GCCTGCCTCT |
| | TTCTCTGCTC | TTTCCACAGA |
| PEA15 | GGAAGACATC | GCACACGGGT |
| | CCCAGCGAAA | TCTGTAGTCA |
| PLCD4 | GGAACTCTGG | AAAGGGCTGA |
| | AATGCAGGCT | TGGGCTTCTC |
| PRDX3 | GCCACATGAA | GGGAGATCGT |
| | CATCGCACTC | TGACGCTCAA |
| PTMA | CGTAGACACC | TTCCTCCCCA |
| | AGCTCCGAAA | CCTTCTTCCT |
| TCF4 | CGAAGGAGGC | GGAGCTAGGG |
| | CTCTTCACAG | AAAGTGCTGG |
| GAPDH | GAAGGTGAAG | GAAGATGGTG |
| | GTCGGAGTC | ATGGGATTTC |

SUPPLEMENTAL TABLE 2
Top 20 genes significantly up- or down-regulated in PC3 JunD KD cells.

| Symbol | Gene Name | Fold Change | P-value |
|---|---|---|---|
| UP | | | |
| CP | ceruloplasmin | 0.925 | 9.480E−03 |
| AKR1C1/AKR1C2 | aldo-keto reductase family 1-member C2 | 0.916 | 3.540E−03 |
| SERPINB4 | serpin family B member 4 | 0.894 | 2.210E−04 |
| CDH1 | cadherin 1 | 0.885 | 1.080E−02 |
| PTGS2 | prostaglandin-endoperoxide synthase 2 | 0.765 | 1.780E−03 |
| ARG2 | arginase 2 | 0.737 | 8.110E−03 |
| SERPINB3 | serpin family B member 3 | 0.736 | 2.870E−03 |
| ARL14 | ADP ribosylation factor like GTPase 14 | 0.712 | 1.000E−02 |
| SERPINE2 | serpin family E member 2 | 0.701 | 8.320E−03 |
| CYP1B1 | cytochrome P450 family 1 subfamily B member 1 | 0.69 | 1.730E−02 |
| CADM2 | cell adhesion molecule 2 | 0.681 | 4.740E−02 |
| CTH | cystathionine gamma-lyase | 0.666 | 1.150E−04 |
| TRIM31 | tripartite motif containing 31 | 0.634 | 1.990E−02 |
| KIAA1551 | KIAA1551 | 0.631 | 2.440E−03 |
| BMP2 | bone morphogenetic protein 2 | 0.631 | 2.720E−02 |
| KDM2A | lysine demethylase 2A | 0.626 | 5.490E−04 |
| CLIC3 | chloride intracellular channel 3 | 0.626 | 1.920E−02 |
| MMP13 | matrix metallopeptidase 13 | 0.612 | 6.320E−03 |
| TBC1D8B | TBC1 domain family member 8B | 0.602 | 1.760E−02 |
| TMEM163 | transmembrane protein 163 | 0.925 | 3.670E−02 |
| Down | | | |
| JunD | JunD proto-oncogene, AP-1 transcription factor subunit | −1.818 | 4.700E−06 |
| PRDX3 | peroxiredoxin 3 | −1.138 | 3.910E−06 |
| EPHA5 | EPH receptor A5 | −0.604 | 1.470E−03 |
| CCNA1 | cyclin A1 | −0.575 | 5.380E−03 |
| NAGA | alpha-N-acetylgalactosaminidase | −0.526 | 3.390E−03 |
| ADRB2 | adrenoceptor beta 2 | −0.497 | 1.310E−02 |
| F2RL2 | coagulation factor II thrombin receptor like 2 | −0.491 | 1.700E−02 |
| CBX4 | chromobox 4 | −0.485 | 1.260E−02 |
| STX6 | syntaxin 6 | −0.479 | 2.040E−02 |
| NDUFAF4 | NADH: ubiquinone oxidoreductase complex assembly | −0.475 | 8.180E−03 |
| | factor 4 | | |
| CMTM7 | CKLF like MARVEL transmembrane domain containing 7 | −0.475 | 1.500E−05 |
| FAM196B | family with sequence similarity 196 member B | −0.462 | 4.880E−04 |
| KCNIP2 | potassium voltage-gated channel interacting protein 2 | −0.445 | 5.030E−03 |
| GNB4 | G protein subunit beta 4 | −0.43 | 6.460E−03 |
| ZBTB46 | zinc finger and BTB domain containing 46 | −0.425 | 2.060E−02 |
| SRD5A1 | steroid 5 alpha-reductase 1 | −0.424 | 8.880E−04 |
| IL12A | interleukin 12A | −0.423 | 4.110E−02 |
| IQCH | IQ motif containing H | −0.417 | 1.840E−02 |
| FOXA2 | forkhead boxA2 | −0.387 | 1.030E−02 |
| NRK | Nik related kinase | −0.374 | 2.680E−02 |

The list includes differentially expressed genes (p < 0.05) that were up-regulated >0.5 or down-regulated <0.5 fold change by JunD KD. The 20 highest and the 20 lowest regulated genes are displayed.

SUPPLEMENTAL TABLE 3
Top 20 proteins significantly up- or down-regulated in PC3 cells JunD KD cells.

| Symbol | Protein Name | Fold Change | P-value |
|---|---|---|---|
| Up | | | |
| NXT1 | NTF2-related export protein 1 | 2.205 | 2.345E−02 |
| PTPN9 | Tyrosine-protein phosphatase non-receptor type 9 | 2.152 | 1.703E−04 |
| DNAAF2 | Protein kintoun | 2.134 | 5.187E−03 |
| GARNL3 | GTPase-activating Rap/Ran-GAP domain-like protein 3 | 2.002 | 2.091E−02 |
| CTAGE5 | cTAGE family member 5 | 1.906 | 9.612E−03 |
| RALGPS2 | Ras-specific guanine nucleotide-releasing factor | 1.755 | 9.634E−06 |
| MFSD12 | Major facilitator superfamily domain-containing protein 12 | 1.630 | 3.639E−02 |
| MYH7 | Myosin-7 | 1.606 | 1.255E−02 |
| UBE2E2 | Ubiquitin-conjugating enzyme E2 | 1.593 | 1.604E−02 |
| DNMT1 | DNA (cytosine-5)-methyltransferase 1 | 1.576 | 2.114E−03 |
| GLOD4 | Glyoxalase domain-containing protein 4 | 1.567 | 1.678E−04 |
| SERPINB6 | Serpin B6 | 1.563 | 8.382E−04 |
| ALMS1 | Alstrom syndrome protein 1 | 1.559 | 2.328E−02 |
| LRCH1 | Leucine-rich repeat and calponin homology domain- | 1.537 | 1.366E−02 |
| | containing protein 1 | | |
| PFN2 | Profilin-2 | 1.531 | 1.443E−05 |
| UBA3 | NEDD8-activating enzyme E1 catalytic subunit | 1.483 | 4.343E−05 |
| ERVFC1 | Endogenous retrovirus group FC1 Env polyprotein | 1.478 | 4.733E−02 |
| TTI2 | TELO2-interacting protein 2 | 1.476 | 6.684E−04 |
| GFRA2 | GDNF family receptor alpha-2 | 1.465 | 4.286E−02 |
| ARMCX3 | Armadillo repeat-containing X-linked protein 3 | 1.461 | 1.792E−02 |
| Down | | | |
| GLS2 | Glutaminase 2 | −4.754 | 1.320E−05 |
| CTSS | Cathepsin S | −3.314 | 4.717E−04 |
| TEX15 | Testis-expressed sequence 15 protein | −3.292 | 3.650E−05 |
| RPF2 | Ribosome production factor 2 homolog | −3.000 | 1.000E−04 |
| TRIM38 | Tripartite motif-containing protein 38 | −2.900 | 5.407E−04 |
| ERO1L | ERO1-like protein alpha | −2.850 | 2.565E−04 |
| ZNF283 | Zinc finger protein 283 | −2.780 | 2.356E−03 |
| FASN | fatty acid synthase | −2.720 | 3.920E−05 |
| CROCC | Rootletin | −2.679 | 5.232E−03 |
| SYNE1 | Nesprin-1 | −2.493 | 2.324E−06 |
| ZBTB44 | Zinc finger and BTB domain-containing protein 44 | −2.459 | 1.625E−03 |
| TMEM62 | Transmembrane protein 62 | −2.437 | 6.932E−06 |
| RHOT2 | Mitochondrial Rho GTPase 2 | −2.416 | 1.476E−02 |
| KIAA2012 | Uncharacterized protein KIAA2012 | −2.406 | 9.850E−06 |
| HSPA8 | Heat shock cognate 71 kDa protein | −2.349 | 4.967E−05 |
| ELMO2 | Engulfment and cell motility protein 2 | −2.253 | 7.269E−05 |
| EHD2 | EH domain-containing protein 2 | −2.242 | 1.581E−03 |
| RNF185 | ring finger protein 185 | −2.192 | 2.505E−02 |

The list includes differentially expressed proteins (p < 0.05) that were up-regulated >1.4 or down-regulated <0.5-fold change by JunD KD. The 20 highest and the 20 lowest regulated proteins are displayed.

SUPPLEMENTAL TABLE 4
JunD-regulated genes: Cell Cycle-related
genes/proteins in PC3 JunD KD cells.

| Gene Symbol | Fold Change | P. Value < 0.05 |
|---|---|---|
| PRDX3 | −1.138 | 3.91E−06 |
| PEA15 | −0.56 | 2.34E−02 |
| STX6 | −0.479 | 2.04E−02 |
| MCMBP | −0.289 | 2.19E−02 |
| KIF2C | −0.288 | 5.60E−03 |
| PRPS2 | −0.279 | 2.19E−03 |
| EML4 | −0.255 | 6.25E−04 |
| SKA3 | −0.25 | 5.32E−02 |
| CTNNA3 | −0.235 | 3.62E−02 |
| ESPL1 | −0.231 | 2.46E−02 |
| CDK2 | −0.223 | 1.32E−03 |
| U2AF1/U2AF1L5 | −0.222 | 3.02E−02 |
| PNN | −0.21 | 2.96E−02 |
| CRIP2 | −0.205 | 1.34E−02 |
| EIF1 | −0.189 | 1.90E−03 |
| SCAMP1 | −0.187 | 3.42E−02 |
| DNMT1 | −0.183 | 4.76E−02 |
| NUP54 | −0.18 | 2.47E−02 |
| ABI2 | −0.178 | 1.42E−02 |
| PKP1 | −0.178 | 2.43E−02 |
| SNRPD1 | −0.177 | 1.88E−02 |
| ASPH | −0.175 | 1.03E−02 |
| SMC1A | −0.174 | 1.25E−03 |
| LIG1 | −0.174 | 2.17E−02 |
| ATP5G1 | −0.162 | 1.02E−02 |
| MGLL | −0.162 | 2.12E−02 |
| TMEM109 | −0.161 | 9.24E−03 |
| EFTUD2 | −0.161 | 3.46E−02 |
| CENPF | −0.157 | 6.18E−03 |
| CDK4 | −0.157 | 2.58E−02 |
| BRIX1 | −0.151 | 2.46E−02 |
| ATP2A2 | −0.146 | 1.89E−02 |
| RHOG | −0.145 | 3.60E−02 |
| FBLIM1 | −0.144 | 2.38E−02 |
| THOC2 | −0.144 | 3.14E−02 |
| FASN | −0.143 | 3.54E−02 |
| RRP1B | −0.14 | 2.17E−02 |
| CKAP5 | −0.136 | 4.07E−02 |
| RPS16 | −0.132 | 1.23E−02 |
| SEC23B | −0.131 | 4.45E−02 |
| TMP0 | −0.127 | 8.20E−03 |
| CBFB | −0.126 | 1.25E−02 |
| CDK1 | −0.122 | 9.40E−03 |
| PRPF40A | −0.119 | 7.57E−03 |
| TRIM28 | −0.113 | 1.57E−02 |
| MYO1C | −0.113 | 3.59E−02 |
| EIF2AK2 | −0.109 | 4.00E−04 |
| DNAJA1 | −0.108 | 1.68E−02 |
| MCM3 | −0.106 | 5.96E−03 |
| RACGAP1 | −0.106 | 2.81E−02 |
| POLR1B | −0.106 | 3.84E−02 |

The list includes shared 75 genes/proteins (cell cycle-related whose expression was significantly down-regulated at p < 0.05 by JunD kD.

SUPPLEMENTAL TABLE 5
JunD target molecules that may contribute to the progression of prostate cancer.

| | Top Diseases and | | Focus | |
|---|---|---|---|---|
| ID | Functions | Score | Molecules | Molecules in Network |
| 1 | Cell Morphology, Cellular | 50 | 23 | 26s Proteasome, ABI2, ASPH, BCAP31, CD3, CENPF, |
| | Function and Maintenance, | | | CRIP2, cytochrome C, DNAJA1, DNMT1, ERCC2, |
| | Tissue Development | | | HISTONE, Histone h3, Histone |
| 2 | Cell Cycle, Cellular Assembly | 33 | 16 | alcohol group acceptor phosphotransferase, ATP5G1, |
| | and Organization, Cellular | | | C/ebp, Calcineurin protein(s), CaMKII, caspase, CD3 |
| | Development | | | group, Cdc2, Cdk, CDK1, CDK2, CDK4, CDK4/6, |
| | | | | Cyclin A, Cyclin D, Cyclin E, E2f, ERK1/2, ESPL1, |
| | | | | FASN, Histone H1, KIF2C, LIG1, MAP2K1/2, MCM3, |
| | | | | MCMBP, PEA15, POLR1B, Rb, Rnr, RPA, RPS5, |
| | | | | RPS28, RRP1B, SMC1A |
| 3 | Protein Synthesis, RNA Post- | 30 | 15 | Akt, ATP2A2, BCR (complex), calpain, CHTOP, Creb, |
| | Transcriptional Modification, | | | DHX36, EIF1, Eif2, EIF2AK2, EIF2S1, ERK, FBLIM1, |
| | Gene Expression | | | Gsk3, IFN Beta, ILF3, Immunoglobulin, Integrin, LDL, |
| | | | | Mek, MGLL, p70 S6k, Pdgf (complex), PDGF BB, |
| | | | | PP2A, RACGAP1, Ras, RHOG, Ribosomal 40s |
| | | | | subunit, RPS6, RPS7, RPS16, Sos, Tgf beta, THOC2 |
| 4 | Cell Cycle, Cellular Movement, | 24 | 13 | ANGEL2, APC, APP, BRIX1, C2orf49, C5orf15, Ck2, |
| | Cell Death and Survival | | | CKAP5, DDX21, DENND1C, DIMT1, DTD2, EGFR, |
| | | | | ELAVL1, EML4, FAM126B, FLOT2, FN1, MPV17L, |
| | | | | MTCP1, MTURN, NUP54, NUP205, PP2D1, PRPS2, |
| | | | | PTPN2, SEC23B, SKA3, SMIM11A, SREBF1, |
| | | | | TCEAL8, TM2D1, tubulin, TXLNG, ZNF573 |
| 5 | RNA Post-Transcriptional | 16 | 9 | CBFB, Cg, CTNNA3, EFTUD2, Gm14277, GNPNAT1, |
| | Modification, Cardiac | | | IgG, IKK (complex), Insulin, Interferon alpha, Jnk, P38 |
| | Arrythmia, Cardiovascular | | | MAPK, p85 (pik3r), PI3K (complex), Pkc(s), PRPF4, |
| | Disease | | | PRPF40A, RNU12, RNU1-2, RNU1-3, RNU1-27P, |
| | | | | RNU1-28P, RNU4-2, RNU5B-1, RNU5D-1, RNU5E-1, |
| | | | | RNU5F-1, RNVU1-7, RNVU1-18, SCAMP1, snRNP, |
| | | | | snRNP-IgG Immune complex, SNRPD1, |
| | | | | U2AF1/U2AF1L5, Vegf |

The list includes shared 76 genes/proteins (cell cycle-related) whose expression was significantly down-regulated at p < 0.05 by JunD KD.

We claim:

1. A method for inhibiting prostate cancer cell growth comprising identifying JunD target genes, decreasing the expression of said target genes, and inhibiting the function of the JunD target genes to interfere with the regulation of cancer cell proliferation and early stages of carcinogenesis.
